(19) United States
(12) Patent Application Publication
(10) Pub. No.: US 2025/0170080 A1
(43) Pub. Date: May 29, 2025

(54) COMPOSITIONS COMPRISING BUTYRIC ACID AND GLUCONIC ACID FOR THE TREATMENT OF DISORDERS AND SYNDROMES ASSOCIATED WITH INTESTINAL BARRIER DYSFUNCTIONS AND DYSBIOSIS

(71) Applicant: DIFASS INTERNATIONAL S.P.A., Coriano (RN) (IT)

(72) Inventors: Alida Agostini, Coriano (RN) (IT); Maria Teresa Sista, Coriano (RN) (IT); Davide Giorgini, Savignano sul Rubicone (FC) (IT)

(73) Assignee: DIFASS INTERNATIONAL S.P.A., Coriano (RN) (IT)

(21) Appl. No.: 18/841,688

(22) PCT Filed: Mar. 1, 2023
(86) PCT No.: PCT/IB2023/051895

§ 371 Date: Aug. 27, 2024

(30) Foreign Application Priority Data
Mar. 2, 2022 (IT) 102022000003929

(51) Int. Cl. A61K 31/19; A61K 31/05; A61K 31/122; A61K 31/14; A61K 31/197; A61K 31/385; A61K 31/4045; A61P 29/00

(52) U.S. Cl. CPC A61K 31/19; A61K 31/05; A61K 31/122; A61K 31/14; A61K 31/197; A61K 31/385; A61K 31/4045; A61P 29/00

(57) ABSTRACT

Disclosed are pharmaceutical or nutraceutical compositions comprising butyric acid or a salt thereof and gluconic acid, which are useful in the treatment of disorders and syndromes associated with intestinal barrier dysfunctions and dysbiosis.

The invention relates to compositions comprising butyric acid or salts thereof and gluconic acid which are useful in the treatment of disorders and syndromes associated with intestinal barrier dysfunctions and dysbiosis.

### PRIOR ART

Intestinal alterations are often associated with a state of low-grade chronic inflammation, which characterises them and further aggravates the state of the intestine, in a vicious circle wherein it is difficult to establish whether the inflammation is the cause or the consequence.

Several factors trigger and feed the low-grade inflammatory state, and are often combined in a multifactorial process:
dietary habits (Western diet, consumption of processed foods, alcohol, etc.)
persistent stress
intestinal dysbiosis (long-term antibiotic or cortisone treatments, prior infections, etc.)
lifestyles (sedentary habits, abuse of laxatives)
alterations in the circadian rhythms.

The persistence of a state of low-grade chronic inflammation alters the intestinal permeability (leaky gut) and the barrier function of the intestine, causes hyperactivation of the intestinal immune system, and gives rise to alterations in the intestinal microbiota, all of which factors lead to a further deterioration in the situation, with local repercussions (constipation, diarrhoea, flatulence, malabsorption) and systemic repercussions (fatigue, reduced immune defences, skin disorders, food intolerances, urinary tract infections and dysmetabolisms) [Konig et al., 2016].

Acute or chronic inflammation and alteration of the intestinal barrier characterise a series of gastrointestinal and other disorders: IBS, IBD, diverticulosis/diverticulitis, food intolerances, recurrent cystitis, psoriasis, atopic dermatitis, alterations in mood and general mental/physical well-being, etc.

Hence the growing interest in researching effective solutions to control and regulate intestinal inflammation processes, the majority whereof involve an attempt to regulate the intestinal microbiota, which is closely connected with inflammation and alteration of permeability.

The largest market is undoubtedly that of probiotics, namely micro-organisms (bacteria, yeasts) which, when consumed in sufficient amounts, perform beneficial functions for the body, mainly by producing metabolites that mediate their beneficial effects.

Probiotics are useful in various situations to compensate for microbial deficiency and rebalance the intestinal flora, restoring normal production of postbiotic metabolites, but some limitations are universally acknowledged: dose, viability, and ability to colonise the intestine, wherein they are required to perform their function and persistence. Attention has recently also focused on other critical factors during the use of probiotics, especially the strain-specific effect (only specific strains are effective in specific situations), and the need to focus more on safety. Integration with probiotic micro-organisms is generally deemed safe, and is recognised as such for most probiotic strains. However, although probiotics can be safe for healthy adults, their use has been associated with a higher risk of infection and/or morbidity in some delicate situations: very low birth-weight babies and infants, adult and child patients in critical conditions in intensive care units, and postoperative, hospitalised or immunocompromised patients [Suez et al., 2019].

Other products on the market promote the use of prebiotic fibres (inulin, FOS, GOS), i.e., dietary fibres that promote the growth and development of intestinal microbiota, with the aim of helping to eliminate dysbiosis and therefore controlling inflammation by indirect mechanisms. There are also numerous products wherein probiotics and prebiotics are combined (synbiotics). There is also a great deal of literature about the use of prebiotic fibres, but once again their limitations are recognised, in particular the need to use them in large amounts (grams) in order for their benefits to be appreciable, the caution required in patients with abdominal pain, because it could be accentuated by promoting fermentation processes, and their aspecific action, as prebiotic fibres can act as nourishment for both “good” and “bad” bacterial flora.

In precarious conditions of the intestinal mucosa (e.g. leaky gut), in particular in the colon, the administration of probiotic bacterial species can be useless, in the absence of adequate conditions for adherence to the wall and replication, and often harmful due to the risk that microbial species may pass through the loose mesh of the intestinal barrier in the underlying tissues, triggering dangerous defense mechanisms that generate an inflammatory state which is first local, and subsequently systemic.

In such cases, even providing prebiotic fibres to stimulate the growth and metabolism of said species may be inadequate, or even counterproductive, because said fibres which, in order to develop a prebiotic effect, must be supplied in units of not less than several grams, generally not less than 5 g, and preferably in doses of 15-20 g a day, can become a nutrient substrate for bacterial species that are harmful to humans. Gluconic acid is also used as a prebiotic, stimulates butyrate production in the large intestine and avoids digestion and absorption in the small intestine, whereas over 70% of the gluconic acid introduced with the diet or diet supplements reaches the large intestine. There, gluconic acid is fermented by various bacteria (Lactobacillus and Bifidobacterium) that form lactate and acetate, which in turn are converted to butyrate by bacteria such as Megasphaera elsdenii. One study conducted on an animal model suggests that a continuous intake of gluconic acid promotes the production of butyrate in the large intestine. These findings indicate that gluconic acid is a useful prebiotic that can contribute to health by producing butyrate in the large intestine. The beneficial health effects of prebiotic compounds obviously depend on the presence of suitable bacterial species to ferment them [Tsukahara et al., 2002; Kameue et al., 2004; Kameue et al., 2006]. Gluconic acid, like other prebiotics, must be taken in amounts of several grams, such as 3 g to 9 g a day, to generate significant effects on human health [Asano et al., 1994].

A recent study conducted on obese patients to evaluate the effects of probiotics and prebiotics, alone and in combination, on the intestinal barrier function, demonstrated that administering probiotics and prebiotics individually improves the barrier function of the intestine, but the combination of probiotics and prebiotics does not generate any obvious synergism [Krumbeck et al., 2018]. Even more recently, a study that retrospectively examined the data of a group of 438 patients who received immunotherapy for metastatic melanoma was published in Science [Spencer et al., 2021]. The therapeutic response to immunotherapy could be radiologically evaluated in 293 patients, and the response was favourable in 193. In this sub-group of “responders”, the 128 with the largest fibre intake in their diet responded best to the treatment. To establish whether there is a cause-effect relationship, fibre intake was experimentally modulated in preclinical models and, as observed in patients, it was confirmed that the group supplied with a diet having a high fibre content responded significantly better to immunotherapy than the control group. However, the most pronounced benefit was observed in patients with a sufficient fibre intake in their diet, without the use of probiotics.

It would therefore be useful to pursue the search for alternative solutions that regulate the inflammatory processes to control intestinal discomfort. A recent option is the use of a postbiotic metabolite, butyrate, a short-chain fatty acid (SCFA) normally produced by fermentation of prebiotic fibres by the intestinal bacteria. Butyrate is the main energy source for the colon cells, meets approximately 60-70% of the energy requirement, and helps to maintain intestinal homeostasis. As regards its regulatory functions, although their action mechanisms have not yet been clarified, butyrate seems able to influence the cell function by means of genetic and epigenetic regulation processes [Fu et al., 2019]. Most of the beneficial effects of probiotics are attributed to butyrate, especially in the control of inflammation and the barrier function. Butyrate is involved in a series of metabolic processes that regulate inflammation: it modulates the resident bacterial flora, improves the epithelial barrier function, and modulates the immune response [Salminen et al., 2021].

One of the mechanisms whereby butyrate can operate as an anti-inflammatory agent is inhibition of NF-κB transcription factor as a result of inhibition of histone deacetylase (HDAC) [Inan et al., 2000]. Butyrate can also directly activate GPCRs (G protein-coupled receptors) to maintain the balance between tolerance for commensal bacteria and immunity to the pathogenic bacteria in the intestinal immune system [Koh et al., 2016; Singh et al., Butyrate also regulates transepithelial ion transport, influences intestinal motility and satiety by increasing expression of peptide YY and proglucagon, and modulates the brain functions by means of epigenetic mechanisms that increase the proportion of intestinal cholinergic neurones. Recent studies demonstrate that butyrate supplementation has beneficial effects on the health, by reducing adipose tissue deposits and increasing insulin sensitivity [Fu et al. 2018].

In order for butyrate taken orally to perform any function, account must be taken of the need for butyrate to be conveyed directly into the colon, where it performs its actions, mainly mediated by the colonocytes. If that were not the case, butyrate would be reused by the cells of other tissues as a readily available energy source. For this reason, colonic release forms which use specific technologies (delayed release, microencapsulation) are preferred. Moreover, butyrate has a rather unpleasant smell and a sour taste, so that technological measures are required to eliminate that problem too.

Prakash A. et al., “Effect of different doses of Manuka honey in experimentally induced inflammatory bowel disease in rats” Physiotherapy Research, 2008, 22 (11), pp 1511-1519, and Aidan G Leong, “Indigenous New Zealand honeys exhibit multiple anti-inflammatory activities”, Innate Immun 2012 June; 18 (3): 459-66, describe the anti-inflammatory properties of Manuka honey, a type of honey derived from the flowers of Leptospermum scoparium. Manuka honey mainly contains polyphenols, together with many other compounds. They include methylglyoxal, which is considered to be one of the ingredients mainly responsible for its well-known antiseptic and anti-inflammatory properties. It also contains over 30 organic acids, including butyric acid. The known activity of Manuka honey obviously cannot suggest the usefulness of a specific combination of butyric acid and gluconic acid in specific ratios.

CN 103 806 773 cites, as ingredients of a food additive, “dibutyric acid sodium di-secondary octyl sulphonate” and “stearic acid gluconolactone”, at least according to the available automatic translation. Said terms are by no means clear, and do not allow the identity of the cited compounds to be established; “stearic acid gluconolactone” appears to be entirely meaningless, while “dibutyric acid sodium di-secondary octyl sulphonate”, according to the best hypothesis, probably refers to “di-sec-octyl sodium dibutyrate sulphonate”. However, even apart from the incorrect terminology, it seems evident that CN 103 806 773 does not describe a combination of butyric acid and gluconic acid in any way.

### DESCRIPTION OF THE INVENTION

The present invention makes available a composition having improved anti-inflammatory activity, using ingredients which are known in the nutritional/dietary field and are therefore deemed safe, having no noteworthy side effects, and which specifically treat acute intestinal disorders whose therapeutic management is complicated by the inflammatory component, even when low-grade. At the same time, the present invention aims to overcome the limitations and critical factors associated with the use of conventional products, which are largely based on the use of probiotic, prebiotic or synbiotic compositions.

Said aims are achieved by the combination in certain ratios of butyric acid or a salt thereof with gluconic acid, giving rise to an unexpected increase in anti-inflammatory activity in the intestine, regardless of the state of eubiosis or different degrees of intestinal dysbiosis.

The object of the invention is therefore pharmaceutical or nutraceutical compositions comprising butyric acid or a salt thereof and gluconic acid or a salt thereof in a weight ratio ranging between 4:1 and 60:1, preferably between 10:1 and 25:1; more preferably between 11:1 and 23:1.

Butyric acid can be used as such or in the form of a salt, such as a sodium, potassium, magnesium or calcium salt. Gluconic acid can also be used “as is” or in the form of a salt.

Typically, in the compositions according to the invention, the weight percent of butyric acid or a salt thereof ranges between 5% and 90%; preferably between 10% and 70%; more preferably between 40% and 60%, and the weight percent of gluconic acid ranges between 0.1% and 8%; preferably between 0.6% and 5%; more preferably between 0.7% and 4%.

The dosage unit of butyric acid or a salt thereof can range from 100 mg to 1,500 mg; preferably from 200 to 1,000 mg; more preferably from 400 mg to 500 mg, while that of gluconic acid can range from 5 mg to 90 mg; preferably from 10 mg to 60 mg; more preferably from 20 mg to 40 mg.

In addition to butyrate and gluconic acid, the composition can also include other ingredients, such as vitamins (A, B group, C, D, E, K), minerals (magnesium, potassium, zinc, iron, calcium, copper, manganese, selenium), plant-based ingredients (extracts, powdered spices, fermentates, oils, essential oils, polyphenols, bioflavonoids), fungi in the form of extracts or powders, algae in the form of extracts or powders, other substances with a nutritional and/or physiological effect (e.g. lipoic acid, melatonin, resveratrol, GABA, coenzyme Q10, choline), essential and non-essential amino acids. The sum of said other ingredients generally ranges between 1% and 15% w/w.

The compositions according to the invention can be formulated in various forms suitable for oral, rectal, buccal or parenteral administration, using conventional techniques and excipients.

Examples of said forms comprise functional foods, foods for special medical purposes, food or diet supplements, pharmaceutical forms such as tablets, hard or soft capsules, granulates, solutions, suspensions, vaginal suppositories, suppositories, creams, gels, enema suspensions.

Additives and excipients suitable to manufacture forms of administration with modified release in various tracts of the intestine can be used.

Advantageously, the compositions according to the invention give rise to an unexpected increase in anti-inflammatory activity, without inducing any undesirable side effects, in particular in the treatment of the disorders and syndromes associated with alterations of the intestinal mucosa, the barrier function of the gastrointestinal tract and dysbiosis, such as irritable bowel syndrome (IBS), chronic inflammatory bowel disease (IBD), diverticulosis, diverticulitis, intestinal infections, coeliac disease, food intolerances, food allergies, constipation, diarrhoea, intestinal alterations and systemic consequences deriving from the use of antibiotics or other medicaments, medical treatments, surgical treatments with an intestinal impact, or food poisoning.

The examples below are illustrative of the invention.

### Examples 1 and 2: Tablets/Coated Tablets

The tablets can be prepared by direct compression of the ingredients or by compression of granular forms thereof (dry or wet granulation). The ingredients can be advantageously divided between two or more layers of the tablet with different ingredient release kinetics.

### Direct Compression

1. Accurately weigh all ingredients in separate containers
2. The pre-weighed ingredients (active ingredients and excipients) are sieved.
3. Mixing of all ingredients
4. Filling of tablet press mould with the mixture prepared in step 3, and formation of the tablet with suitable compressive force.

### Compression of Granular Forms

### Dry Granulation

1. Accurately weigh all ingredients to be granulated in separate containers
2. The pre-weighed ingredients (active ingredients and excipients) are sieved.
3. Mixing of the ingredients with suitable pre-weighed agents, such as binders, diluents, etc., to promote granule formation by means of compacting and fragmentation.
4. Proceed with the optional sieving step of the granules obtained.
5. Mixing of the resulting granulates with other ingredients, if any.
6. Filling of tablet press mould with the mixture prepared in step 5, and formation of the tablet with suitable compressive force.

### Wet Granulation

1. Accurately weigh all ingredients to be granulated.
2. The pre-weighed ingredients (active ingredients and excipients) are optionally sieved.
3. Mixing of the ingredients with the excipients, if any, to be inserted in the rack of the fluid-bed granulator.
4. Preparation of granulating solution: in a separate container, weigh the binders and/or coating agents and disperse them in a suitable solvent to obtain a clear solution.
5. Transfer the solution to the granulator and start the granulation process under controlled conditions.
6. Proceed with the drying step at a controlled temperature (about 50° C.±5° C.), eliminating the solvent.
7. Proceed with the optional sieving step of the granules obtained.
8. Mixing of the resulting granulates with other ingredients, if any.
9. Filling of tablet press mould with the mixture prepared in step 8, and formation of the tablet with suitable compressive force.

### Preparation of Coating

1. Accurately weigh all coating ingredients and a suitable amount of solvent or mixture of solvents.
2. Combine the ingredients gradually with the solvent until homogenisation is complete.
3. Filter the solution through suitable filters.
4. Transfer the tablets to a specific coating pan and start the film-coating process under suitable temperature, pressure and time conditions.

### Example 1

| | % w/w |
|---|---|
| Core | |
| Sodium butyrate | 22.73% |
| Gluconic acid | 1.40% |
| Other active ingredients | 15.00% |
| Sorbitol | 29.26% |
| Microcrystalline cellulose | 21.80% |
| Magnesium stearate | 1.50% |
| Silicon dioxide | 1.17% |
| Stearic acid | 0.27% |
| Hydroxypropyl methylcellulose | 0.01% |
| Coating | |
| Shellac | 2.28% |
| Hydroxypropyl methylcellulose | 3.13% |
| Microcrystalline cellulose | 0.27% |
| Stearic acid | 0.27% |
| Titanium dioxide | 0.41% |
| Glycerol | 0.30% |
| Mint flavouring | 0.20% |
| TOTAL TABLET | 100.00% |

### Example 2

| | % w/w |
|---|---|
| Core | |
| Sodium butyrate | 46.05% |
| Gluconic acid | 2.75% |
| Other active ingredients | 1.76% |
| Sorbitol | 24.41% |
| Microcrystalline cellulose | 14.84% |
| Magnesium stearate | 1.47% |
| Silicon dioxide | 1.15% |
| Stearic acid | 0.26% |
| Hydroxypropyl methylcellulose | 0.01% |
| Coating | |
| Shellac | 2.24% |
| Hydroxypropyl methylcellulose | 2.49% |
| Magnesium carbonate | 1.08% |
| Calcium phosphate | 0.68% |
| Microcrystalline cellulose | 0.09% |
| Stearic acid | 0.09% |
| Glycerol | 0.30% |
| Mint flavouring | 0.33% |
| TOTAL TABLET | 100.00% |

### Example 3: Rigid Capsules

The capsules can be prepared by direct filling of the shells with the ingredients in the form of powders or in the form of granules or microgranules (obtained by dry or wet granulation).
1. Accurately weigh all ingredients (active ingredients, excipients) in the form of fine powder or in the form of granulates or microgranules.
2. Mix all ingredients.
3. Fill capsules with the mixture obtained in step 2, and close.
4. Packaging of capsules after favourable outcome of conformity controls.

| | % w/w |
|---|---|
| Sodium butyrate | 41.67% |
| Gluconic acid | 2.49% |
| Other active ingredients | 4.18% |
| Hydrogenated palm oil | 10.42% |
| Dibasic calcium phosphate | 21.91% |
| Magnesium stearate | 2.00% |
| Silicon dioxide | 1.00% |
| Capsule | 16.33% |
| TOTAL CAPSULE | 100.00% |

### Example 4: Sachets/Stick Packs Containing Water-Dispersible and/or Orodispersible Powders and/or Granulates

Ingredients in the form of powders and/or the subform of granulates or microgranules are directly dispensed into in sachets/stick packs.
1. Accurately weigh all ingredients (active ingredients, excipients).
2. Mix all ingredients.
3. Fill the preformed packaging of the sachets or stick packs with the mixture obtained in step 3.
4. Packaging of sachets or stick packs after favourable outcome of conformity controls.

| | % w/w |
|---|---|
| Calcium butyrate | 12.50% |
| Gluconic acid | 0.75% |
| Other active ingredients | 10.09% |
| Hydrogenated palm oil | 3.14% |
| Sorbitol | 23.96% |
| Fructose | 48.33% |
| Silicon dioxide | 0.85% |
| Sucralose | 0.06% |
| Mint flavouring | 0.32% |
| TOTAL WEIGHT OF | 100.00% |
| SACHET | |

### Example 5: Soft Capsules

Before the capsule-filling process begins, two preparatory processes are usually conducted simultaneously but separately, leading to the formation of the two parts of a soft capsule, i.e., manufacture of (1) the gel mass and (2) the matrix which acts as filler for said capsules.

### 1. Manufacture of Gel Mass

a. Dissolve under vacuum the gelatin previously weighed in water at about 80° C., then add a plasticiser (e.g. glycerol).
b. When the gelatin has completely solubilised, the other pre-weighed ingredients, such as colourings, opacifiers and flavouring substances, are added.
c. The hot gelatin mass is transferred in two separate ribbons, each about 150 mm long, where they undergo a sol-gel transition of controlled thickness due to cooling. Each ribbon provides half the soft capsule, and each half is transferred to the capsule-filling system.

### 2. Manufacture of Liquid Filling Matrix

a. Accurately weigh all ingredients (active ingredients, excipients), including the liquid carrier.
b. Sieve the pre-weighed ingredients to ensure that their particle size does not exceed 200 μm.
c. Mix/disperse the sieved ingredients in the liquid carrier with a homogeniser-mixer.
3. Accurately measured volumes of liquid filler are injected into the space between the two gelatin ribbons at a temperature of about 40° C. The two half-capsules are sealed by applying heat and pressure.
4. The capsules thus formed are placed in a dryer and passed through an air dryer with 20% relative humidity.
5. Packaging after favourable outcome of conformity controls.

| | % w/v |
|---|---|
| Calcium butyrate | 13.33% |
| Gluconic acid | 0.80% |
| Other active ingredients | 14.83% |
| Lecithin | 2.40% |
| Glycerol | 49.68% |
| Gelatin | 18.74% |
| Iron oxides | 0.22% |
| TOTAL WEIGHT OF | 100.00% |
| SOFTGEL | |

### Example 6: Solutions/Suspensions

Dispersion of the ingredients in an aqueous or non-aqueous liquid phase gives rise to solutions or suspensions, depending on the degree of solubility of the individual ingredients. The solutions may be intended for oral or non-oral use.
1. Accurately weigh all ingredients (active ingredients, excipients).
2. Solubilise/disperse in the aqueous or non-aqueous liquid carrier, generally in increasing order of solubility of the ingredients
3. Package in a suitable container after favourable outcome of conformity controls.

| | % w/v |
|---|---|
| Sodium butyrate | 23.81% |
| Gluconic acid | 1.42% |
| Other active ingredients | 5.00% |
| Honey | 13.89% |
| Glycerol | 37.30% |
| Flavouring | 0.56% |
| Water | 18.02% |
| TOTAL WEIGHT OF SOLUTION | 100.00% |

### Example 7: Evaluation of Anti-Inflammatory Properties

To evaluate their anti-inflammatory properties, the compounds described, forming the object of the present invention, were examined both individually and combined, using an experimental model. In the study described, the following active ingredients and combinations were compared on a reconstructed human intestinal tissue model to evaluate their efficacy in modulating the intestinal inflammatory response:
calcium butyrate
gluconic acid
gluconic acid+calcium butyrate

Assays of pro-inflammatory cytokines TNF-alpha and IL-6 were evaluated. The biological model used in the assay is a model of human intestinal epithelium reconstructed in vitro in 3D, consisting of intestinal epithelial and endothelial cells and fibroblasts of human origin. The highly differentiated tissue model is produced at the air-liquid interface in easily manageable culture tissue inserts using a serum-free medium. Ultrastructural analysis demonstrates the presence of tight junctions, and the mucus-secreting granules imitate the tissue in vivo. The cells used to produce the tissue model were screened for potential biological contaminants, using specific tests and analyses to guarantee the functionality and quality of the tissues. The experimental intestinal epithelium model is therefore devoid of biological contamination, and does not exhibit populations of bacteria or micro-organisms in general. Thus all the findings of the present study must be deemed to be wholly independent of the effects of bacteria (probiotic effects), whether they belong to the normal intestinal bacterial flora, commensal, pathobiontic or pathogenic bacteria, and therefore also independent of the effects of fibres or other prebiotic substances used by bacteria (prebiotic effects).

The intestinal epithelium tissues reconstructed in vitro were exposed for 24 hours to the stressor lipopolysaccharide of bacterial origin (LPS) in combination with cytokine TNF-alpha, and simultaneously treated for a further 24 hours with each experimental condition examined. After the 48-hour experimental session, the culture media were harvested to assay pro-inflammatory cytokines TNF-alpha and IL-6.

Each ingredient and the mixture selected for the treatments were diluted in H₂O, taking account of the average intestinal volume of 80 ml. In particular, the following reference doses were used:
calcium butyrate 500 mg;
gluconic acid 29.87 mg;
gluconic acid+calcium butyrate=29.87 mg+500 mg.

50 μl of each sample was tested in the tissue model, simulating the following experimental conditions:
reference dose;
   The experimental protocol involved:
untreated tissues (CTR−);
tissues exposed to pro-inflammatory agents LPS+TNF-alpha (CTR+);
tissues exposed to pro-inflammatory agents LPS+TNF-alpha and treated with butyrate (calcium butyrate);
tissues exposed to pro-inflammatory agents LPS+TNF-alpha and treated with gluconic acid;
tissues exposed to pro-inflammatory agents LPS+TNF-alpha and treated with gluconic acid+butyrate (calcium butyrate) mixture;
Each test was conducted in duplicate. To monitor the ability of the tested ingredients to inhibit the inflammatory response in vitro in a system wherein an inflammatory condition has been induced, the production of pro-inflammatory cytokine TNF-alpha and interleukin 6 (IL-6) was evaluated. At the end of the treatments the culture media were harvested for the TNF-alpha and IL-6 assay using an ELISA kit. The data obtained were subjected to statistical analysis and compared using the t-test. The variations compared with the positive control are deemed significant for p<0.05 and indicated by “*”.

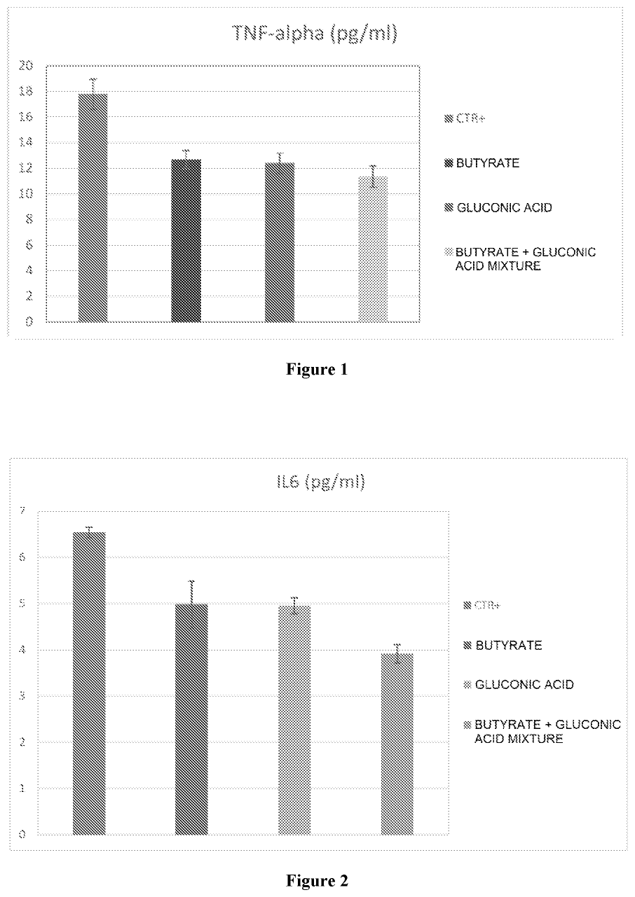

The TNF-alpha levels in CTR−, CTR+ and the tissues treated with the experimental protocol are illustrated in Table 1 and FIG. 1. The results are expressed as mean values ±standard deviation (expressed in pg/ml), as % variation compared with CTR− and as anti-inflammatory activity compared with CTR+. Analysis of the TNF-alpha levels demonstrated that treatments with butyrate, gluconic acid and the mixture (butyrate+gluconic acid) significantly reduce the release of the pro-inflammatory cytokine, and therefore of the inflammatory state in response to induced stress.

TABLE 1

| | Mean | SD | % var. | Anti-inflammatory |
|---|---|---|---|---|
| TNF-alpha | (pg/ml) | (pg/ml) | vs CTR − | activity |
| CTR − | 9.01 | 1.36 | | |
| CTR + | 17.79 | 1.17 | 97.3% | |
| Butyrate | 12.67 | 0.71 | 40.5% | 28.8%* |
| Gluconic acid | 12.40 | 0.79 | 37.6% | 30.3%* |
| Butyrate + gluc. ac. | 11.34 | 0.84 | 25.8% | 36.3%* |

The IL-6 levels in CTR−, CTR+ and tissues treated according to the experimental protocol are illustrated in Table 2 and FIG. 2. The results are expressed as mean values ±standard deviation (expressed in pg/ml), as % variation compared with CTR− and as anti-inflammatory activity compared with CTR+. Analysis of the IL-6 levels demonstrated that the treatments with butyrate, gluconic acid and the mixture (butyrate+gluconic acid) significantly reduce release of the pro-inflammatory cytokine, and therefore of the inflammatory state in response to induced stress.

TABLE 2

| | Mean | SD | % var. vs | Anti-inflammatory |
|---|---|---|---|---|
| IL-6 | (pg/ml) | (pg/ml) | CTR − | activity |
| CTR − | 3.09 | 0.20 | | |
| CTR + | 6.54 | 0.11 | 112.0% | |
| Butyrate | 4.98 | 0.50 | 61.5% | 23.8%* |
| Gluconic acid | 4.95 | 0.17 | 60.5% | 24.3%* |
| Butyrate + gluc. ac. | 3.91 | 0.20 | 26.9% | 40.2%* |

The tests conducted clearly demonstrate the anti-inflammatory effect of butyrate, as described in the literature. Gluconic acid is known for its prebiotic effects and the resulting benefits for the intestinal flora and the host. No other effects have ever been demonstrated or postulated. The tests conducted clearly demonstrate the primary anti-inflammatory effect of gluconic acid, which is not secondary to and/or dependent on the prebiotic effect, as the in vitro model does not exhibit bacterial populations or those normally present in the human or animal intestine, or pathogens or contaminants of any kind, whether viral or fungal. The effect observed is therefore unexpected. The anti-inflammatory effect of the butyrate+gluconic acid mixture is surprising; it is superior to that of the individual ingredients, as shown in bold type in Tables 1 and 2, an effect which was not obvious, since interferences in this specific ambit have been documented.

### REFERENCES

Asano T, Yuasa K, Kunugita T, Teraji T, Mitsuoka T. Effects of gluconic acid on human faecal bacteria. Microbial Ecology in Health and Disease vol. 7:247-256 (1994).
Fu X, Liu Z, Zhu C, Mou H, Kong Q. Nondigestible carbohydrates, butyrate, and butyrate-producing bacteria. Crit Rev Food Sci Nutr. 2019; 59 (sup1): S130-S152.
Inan M S, Rasoulpour R J, Yin L, Hubbard A K, Rosenberg D W, Giardina C. The luminal short-chain fatty acid butyrate modulates N F-kappaB activity in a human colonic epithelial cell line. Gastroenterology. 2000 April; 118 (4): 724-34.
Kameue, C., Tsukahara, T., Yamada, K., Koyama, H., Iwasaki, Y., Nakayama, K., and Ushida, K., Dietary sodium gluconate protects rats from large bowel cancer by stimulating butyrate production. J. Nutr., 134, 940-944 (2004).
Kameue, C., Tsukahara, T., Ushida, K., Alteration of gene expression in the colon of colorectal cancer model rat by dietary sodium gluconate. Biosci Biotechnol Biochem. 2006 March; 70 (3): 606-14.
Koh A, De Vadder F, Kovatcheva-Datchary P, Bäckhed F. From Dietary Fiber to Host Physiology: Short-Chain Fatty Acids as Key Bacterial Metabolites. Cell. 2016 Jun. 2; 165 (6): 1332-1345.
König J, Wells J, Cani P D, García-Ródenas C L, MacDonald T, Mercenier A, Whyte J, Troost F, Brummer R J. Human Intestinal Barrier Function in Health and Disease. Clin Transl Gastroenterol. 2016 Oct. 20; 7 (10): e196.
Krumbeck J A, Rasmussen H E, Hutkins R W, et al. Probiotic Bifidobacterium strains and galactooligosaccharides improve intestinal barrier function in obese adults but show no synergism when used together as synbiotics. Microbiome. 2018; 6 (1): 1-16.
Salminen S, Collado M C, Endo A, Hill C, Lebeer S, Quigley E M M, Sanders M E, Shamir R, Swann J R, Szajewska H, Vinderola G. The International Scientific Association of Probiotics and Prebiotics (ISAPP) consensus statement on the definition and scope of postbiotics. Nat Rev Gastroenterol Hepatol. 2021 September; 18 (9): 649-667.
Spencer C N, McQuade J L, Gopalakrishnan V, McCulloch J A, Vetizou M, Cogdill A P, Khan M A W, Zhang X, White M G, Peterson C B, et al. Dietary fiber and probiotics influence the gut microbiome and melanoma immunotherapy response. Science. 2021; 374 (6575): 1632-1640.
Salminen S, Collado M C, Endo A, Hill C, Lebeer S, Quigley E M M, Sanders M E, Shamir R, Swann J R, Szajewska H, Vinderola G. The International Scientific Association of Probiotics and Prebiotics (ISAPP) consensus statement on the definition and scope of postbiotics. Nat Rev Gastroenterol Hepatol. 2021 September; 18 (9): 649-667.
Singh N, Gurav A, Sivaprakasam S, Brady E, Padia R, Shi H, Thangaraju M, Prasad P D, Manicassamy S, Munn D H, Lee J R, Offermanns S, Ganapathy V. Activation of Gpr109a, receptor for niacin and the commensal metabolite butyrate, suppresses colonic inflammation and carcinogenesis. Immunity. 2014 Jan. 16; 40 (1): 128-39.
Suez J, Zmora N, Segal E, Elinav E. The pros, cons, and many unknowns of probiotics. Nat Med. 2019 May; 25 (5): 716-729.
Tsukahara, T., Koyama, H., Okada, M., and Ushida, K., Stimulation of butyrate production by gluconic acid in batch culture of pig cecal digesta and identification of butyrate-producing bacteria. J. Nutr., 132, 2229-2234 (2002).

1. Pharmaceutical or nutraceutical compositions comprising butyric acid or a salt thereof and gluconic acid in a weight ratio ranging from 4:1 to 60:1.

2. The pharmaceutical or nutraceutical compositions according to claim 1 wherein butyric acid is in the form of a sodium, potassium, magnesium or calcium salt.

3. The pharmaceutical or nutraceutical compositions according to claim 1, wherein the weight percent of butyric acid or a salt thereof ranges from 5% to 90%.

4. The pharmaceutical or nutraceutical compositions according to claim 1, wherein the weight percent of gluconic acid ranges from 0.1% to 8%.

5. The pharmaceutical or nutraceutical compositions according to claim 1, comprising 100 mg to
1,500 mg per dosage unit of butyric acid or a salt thereof.

6. The pharmaceutical or nutraceutical compositions according to claim 1, comprising 5 mg to 90 mg of gluconic acid per dosage unit.

7. The pharmaceutical or nutraceutical compositions according to claim 1, further comprising one or more ingredients selected from vitamins, minerals, herbal derivatives in the form of extracts or powders, fermentates, oils, essences, polyphenols, bioflavonoids, fungi in the form of extracts or powders; algae in the form of extracts or powders; lipoic acid, melatonin, resveratrol, GABA, coenzyme Q10, choline and amino acids.

8. The pharmaceutical or nutraceutical compositions according to claim 1, in the form of tablets, hard or soft capsules, granulates, solutions, suspensions, vaginal suppositories, suppositories, creams, gels, or enema suspensions.

9. A method of treating inflammatory bowel conditions in patients in need thereof with the pharmaceutical or nutraceutical compositions according to claim 1, said method comprising
administering to said patients a pharmaceutical effective amount of said compositions.

10. The method according to claim 9, wherein said inflammatory bowel conditions comprise chronic inflammatory bowel disease, irritable bowel syndrome, diverticulosis and diverticulitis, intestinal changes and disorders due to drug or antibiotic treatment, intestinal infections, leaky gut syndrome and food intolerances.

11. The pharmaceutical or nutraceutical compositions according to claim 1, wherein the weight ratio ranges from 10:1 to 25:1.

12. The pharmaceutical or nutraceutical compositions according to claim 1, wherein the weight ratio ranges from 11:1 to 23:1.

13. The pharmaceutical or nutraceutical compositions according to claim 3, wherein the weight percent of butyric acid or a salt thereof ranges from 10% to 70%.

14. The pharmaceutical or nutraceutical compositions according to claim 3, wherein the weight percent of butyric acid or a salt thereof ranges from 40% to 60%.

15. The pharmaceutical or nutraceutical compositions according to claim 4, wherein the weight percent of gluconic acid ranges from 0.6% to 5%.

16. The pharmaceutical or nutraceutical compositions according to claim 4, wherein the weight percent of gluconic acid ranges from 0.7% to 4%.

17. The pharmaceutical or nutraceutical compositions according to claim 5, comprising 200 mg to 1000 mg per dosage unit of butyric acid or a salt thereof.

18. The pharmaceutical or nutraceutical compositions according to claim 5 comprising 400 mg to 500 mg per dosage unit of butyric acid or a salt thereof.

19. The pharmaceutical or nutraceutical compositions according to claim 6, comprising 10 mg to 60 mg of gluconic acid per dosage unit.

20. The pharmaceutical or nutraceutical compositions according to claim 6, comprising 20 mg to 40 mg of gluconic acid per dosage unit.
